(19) United States
(12) Patent Application Publication
(10) Pub. No.: US 2017/0333565 A1
(43) Pub. Date: Nov. 23, 2017

(54) TLR9 TARGETED CYTOTOXIC AGENTS

(71) Applicant: H. LEE MOFFITT CANCER CENTER AND RESEARCH INSTITUTE, INC., TAMPA, FL (US)

(72) Inventors: ALAN F. LIST, TAMPA, FL (US); SHENG WEI, TAMPA, FL (US)

(21) Appl. No.: 15/522,956

(22) PCT Filed: Oct. 30, 2015
(86) PCT No.: PCT/US15/58315

§ 371 Date: Apr. 28, 2017

Related U.S. Application Data
(60) Provisional application No. 62/073,806, filed on Oct. 31, 2014.

(51) Int. Cl. A61K 47/54; A61K 51/06; A61K 38/08; A61K 31/713; G01N 33/574; C12N 15/113

(52) U.S. Cl. CPC A61K 47/549; G01N 33/57407; C12N 15/113; A61K 38/08; A61K 31/713; A61K 51/065; C12N 2310/14; C12N 2310/3519

(57) ABSTRACT

Disclosed are compositions and methods for targeted treatment of TLR9-expressing cancers. In particular, molecules containing a TLR9 targeting ligand, such as a CpG oligodeoxynucleotide, that target cytotoxic agents to TLR9-expressing malignant cells are disclosed. Compositions and methods are disclosed for targeted treatment of cancer or cancer-stem cells with extracellular TLR9 expression, such as primary human MDS progenitors and hematopoietic stem cell (HSC). In particular, molecules containing TLR9 targeting ligands that target cytotoxic agents to TLR9-expressing malignant cells are disclosed.

### CROSS-REFERENCE TO RELATED APPLICATIONS

This application claims benefit of U.S. Provisional Application No. 62/073,806, filed Oct. 31, 2014, which is hereby incorporated herein by reference in its entirety.

### TECHNICAL FIELD

This application relates generally to compositions and methods for treating cancers, such as myelodysplastic syndromes (MDS).

### BACKGROUND

Myelodysplastic syndromes (MDS) are hematopoietic stem cell malignancies with a rising prevalence owing to the aging of the American population. MDS comprise a group of malignant hematologic disorders associated with impaired erythropoiesis, dysregulated myeloid differentiation and increased risk for acute myeloid leukemia (AML) transformation. The incidence of MDS is increasing with 15,000 to 20,000 new cases each year in the United States and large numbers of patients requiring chronic blood transfusions. Ineffective erythropoiesis remains the principal therapeutic challenge for patients with more indolent subtypes, driven by a complex interplay between genetic abnormalities intrinsic to the MDS clone and senescence dependent inflammatory signals within the bone marrow (BM) microenvironment. Although three agents are approved for the treatment of MDS in the United States (US), lenalidomide (LEN) represents the only targeted therapeutic. Treatment with LEN yields sustained red blood cell transfusion independence accompanied by partial or complete resolution of cytogenetic abnormalities in the majority of patients with a chromosome 5q deletion (del5q), whereas only a minority of patients with non-del5q MDS achieve a meaningful response, infrequently accompanied by cytogenetic improvement. Although responses in patients with del5q MDS are relatively durable, lasting a median of 2.5 years, resistance emerges over time with resumption of transfusion dependence.

The available effective treatment options for patients with non-del(5q) is limited. Notably, MDS cases grow year over year due the increase in the American aging population and its combination. Frequently they are misdiagnosed leading to failure to treat serious infections or the wasting of expensive treatment and precious resources. Once a proper diagnosis is made patients have to rely on frequent blood transfusion and non-specific chemotherapy which have severe side effects and have limited benefit for patients with non-del(5q). The lack of effective treatment on MDS patients without del(5q) contributes to the enormous burden of this disease on both patient and caregivers and increases the risk of AML transformation. Therefore, there is definitely a need to develop a specific targeted therapeutic in this patient population.

### SUMMARY

Compositions and methods are disclosed for targeted treatment of cancer or cancer-stem cells with extracellular TLR9 expression, such as primary human MDS progenitors and hematopoietic stem cell (HSC). In particular, molecules containing TLR9 targeting ligands that target cytotoxic agents to TLR9-expressing malignant cells are disclosed.

In some embodiments, the molecule is defined by the formula:

TTL-CA,

wherein “TTL” represents the TLR9 targeting ligand,

wherein “CA” represents the cytotoxic agent, and

wherein “-” represents a bivalent linker.

In a variety of aspects, the TLR9 targeting ligand is an unmethylated CpG oligodeoxynucleotide, or an analogue or derivative thereof that binds TLR9.

In some embodiments, the cytotoxic agent is a lytic peptide. For example, the lytic peptide can comprise the amino acid sequence PNPNNNPNPN (SEQ ID NO:48), wherein “P” is any polar amino acid, and wherein “N” is any non-polar amino acid. In some cases, the lytic peptide comprises the amino acid sequence KIKMVISWKG (SEQ ID NO:1).

In some embodiments, the cytotoxic agent comprises a functional nucleic acid that is cytotoxic to cancer cells. For example, the functional nucleic acid can inhibit anti-apoptotic gene targets, e.g., anti-apoptotic Bcl-2 member proteins. The functional nucleic acid can also inhibit targets causing drug sensitization (e.g., PP2A and CDC25c). In some cases, the cytotoxic agent comprises a functional nucleic acid that promotes apoptotic gene targets, e.g., apoptotic Bcl-2 member proteins.

Also disclosed is a pharmaceutical composition comprising a molecule disclosed herein in a pharmaceutically acceptable carrier. Also disclosed is a method for treating a TLR9-positive cancer in a subject that involves administering to the subject a therapeutically effective amount of a disclosed pharmaceutical composition. In some cases, the cancer comprises a myelodysplastic syndrome (MDS). For example, the cancer can be non-del(5q) MDS. FIG. 4 identifies other cancers, such as lung and breast cancers, that have increased TLR9 expression. FIG. 5 shows TLR9 protein expression in a variety of tumor tissues. For example, cancers of the skin, esophagous, colon, rectum, liver, lung, and uterus have been shown to have increased TLR9 protein expression. In some cases, the method further involves assaying a biopsy sample from the subject for TLR9 expression prior to treatment.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing cell death (%) of TLR9 transfected HEK cells as a function of CpG-lytic peptide dosage over 48 hours before measuring cell death by flow cytometry. Dotted line represents control TLR9 negative cells with 10 μg/ml of the CpG-lytic peptide.

FIG. 2 is a bar graph showing cell death (% Annexin V) of cells transfected with control vector or TLR9 vector and treated with control peptide or varying dosages of CpG lytic peptide.

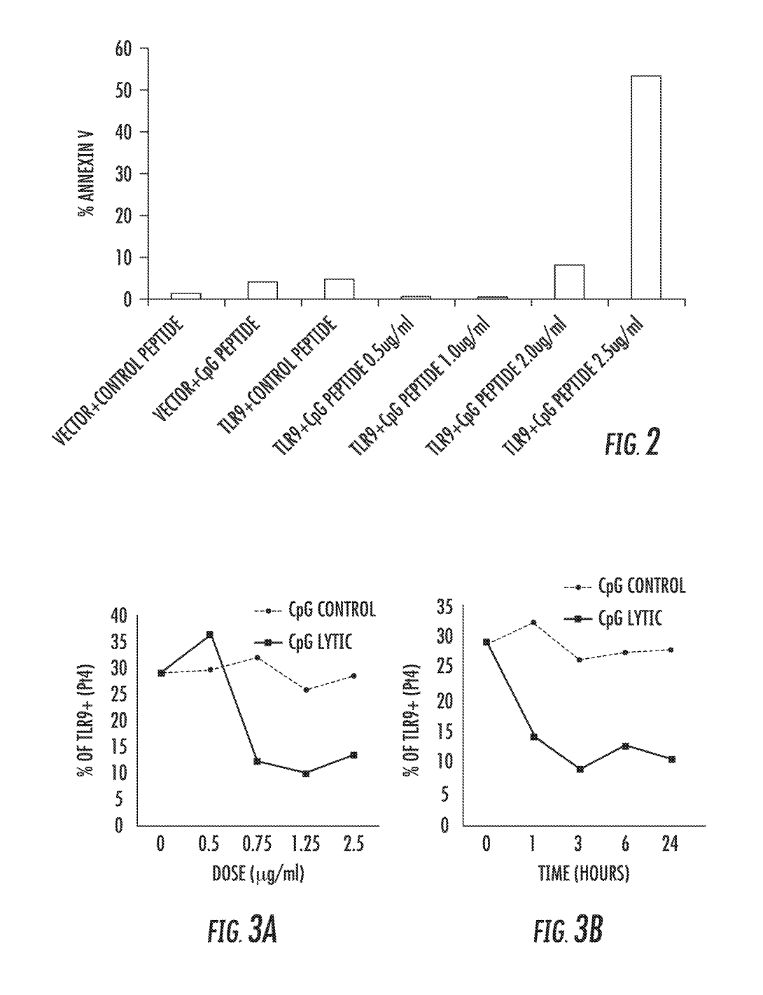

FIGS. 3A to 3C show treatment with CpG lytic peptide depletes TLR9 membrane expressing cells in primary specimens. FIG. 3A is a graph showing MDS-BM samples treated with increasing concentrations of either the CpG lytic peptide or CpG control for 48 hours (representative of four patient samples tested and measured for changes in surface TLR9+ population). FIG. 3B is a graph showing MDS-BM samples treated with 2.5 μg/ml CpG lytic peptide or CpG control at different time points. FIG. 3C is a bar graph showing CpG lytic treatment (2.5 μg/ml) of splenocytes from either wild type or S100A9Tg mice which represents a murine model of MDS.

FIG. 4 is a box plot showing TLR9 overexpression in a variety of tumors. The box plot represents the 25th to 75th percentile (the box) with the median represented by the black line in the box. The outliers are in circles represent the median absolute deviation (2 SD is about the same).

FIG. 5 shows results of tissue microarray (TMA) slides being stained with anti-TLR9 antibody. The top graph shows the tabulated data for the cores in the control TMA and a representative picture from one of the cores demonstrating the lack of brown coloration. The only positive core in that slide was inflamed tonsils which serve as a positive control. The second graph shows the tabulated results from the multi-tumor TMA (48 cases of 15 cancers) showing varied levels of TLR9 positive staining. The picture represents the core for a melanoma case that had heavy brown staining as a representative figure.

FIG. 6 shows an example of MDS BM patient specimen treated with the si-MCL-1 linked CpG demonstrating reduction of TLR9 positive cells after in vitro culture. Non target=non-targeting control; NoTx=no treatment control.

FIGS. 7A and 7B are CT and Pet Scans of the MDS murine model S100A9Tg mice (FIG. 7B) showing increased uptake of CpG linked conjugates compared to normal wildtype controls (FIG. 7A). Mice were injected with 90 μCi-CpG of F18 by tail vein and imaged by CT and Pet scan after at least 60 min.

### DETAILED DESCRIPTION

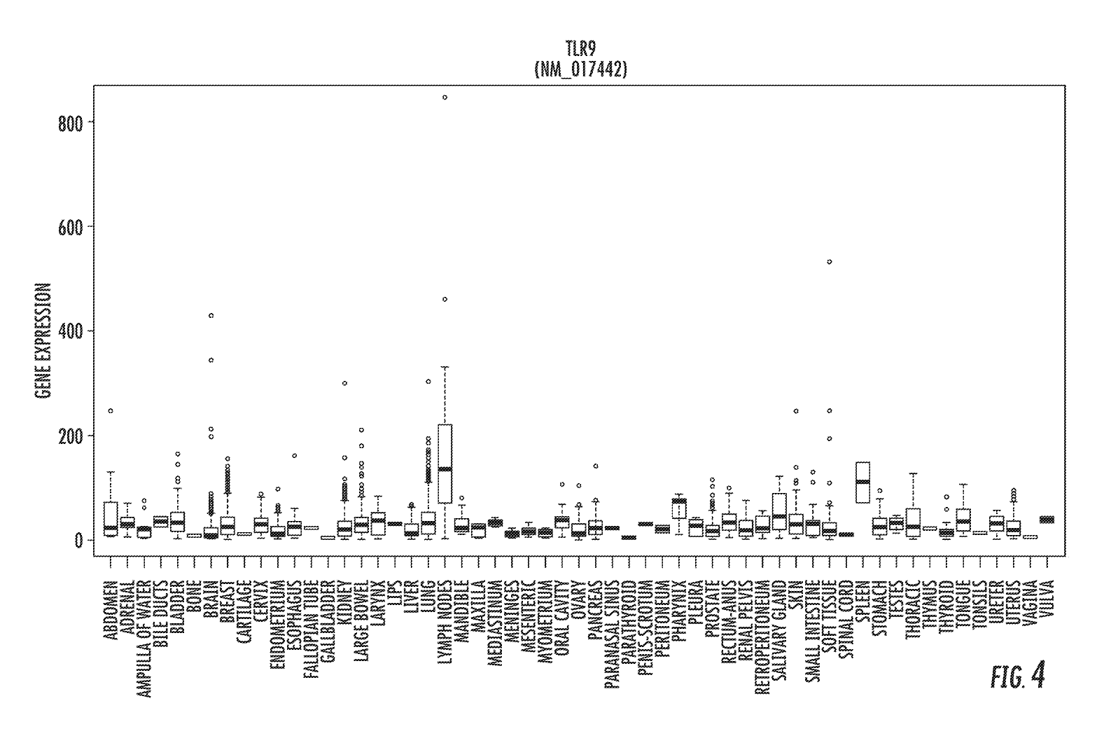

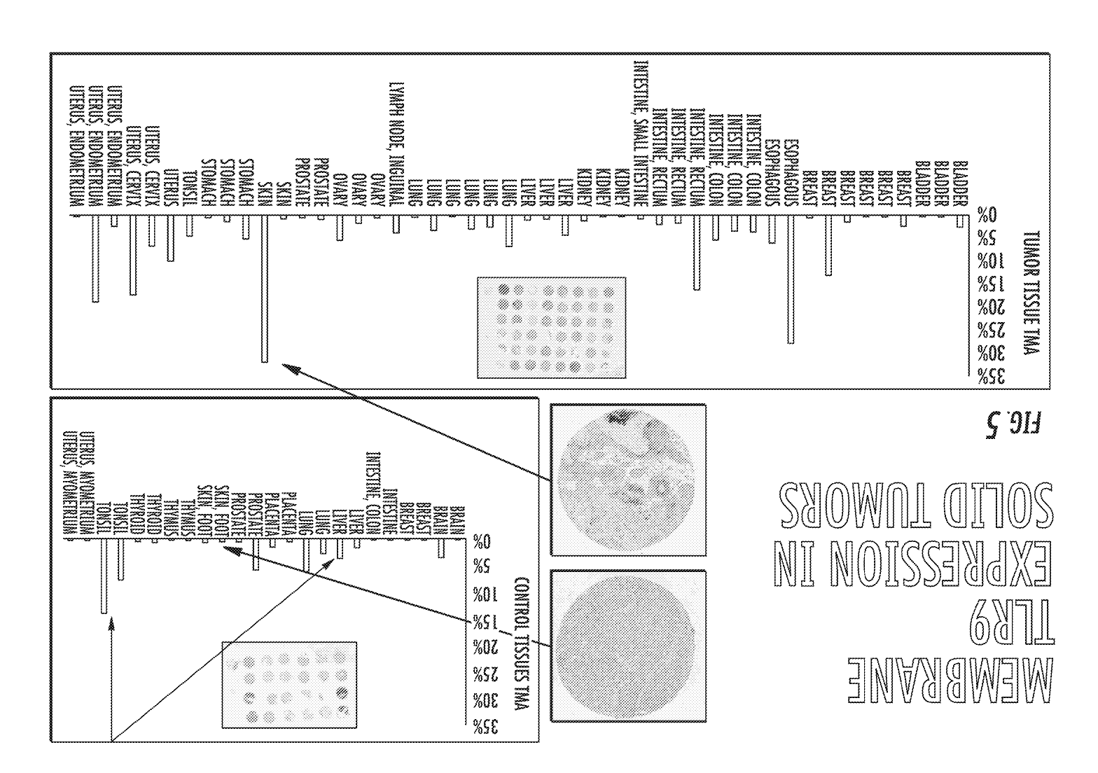

Disclosed are compositions and methods for targeted treatment of TLR9-expressing cancers, such as primary human MDS hematopoietic stem and progenitor cells (HSPC). FIG. 4 identifies other cancers, such as lung and breast cancers, that have increased TLR9 expression. FIG. 5 shows TLR9 protein expression in a variety of tumor tissues. For example, cancers of the skin, esophagous, colon, rectum, liver, lung, and uterus have been shown to have increased TLR9 protein expression.

In particular, molecules containing TLR9 targeting ligands that target cytotoxic agents to TLR9-expressing malignant cells are disclosed. Therefore, the molecule can comprise a TLR9 targeting ligand (“TTL”) and a cytotoxic agent. For example, the TTL and cytotoxic agent can be joined by a bivalent linker.

TLR9 Targeting Ligand

The TTL can in some embodiments be a CpG oligodeoxynucleotide, such as an unmethylated CpG oligodeoxynucleotide, or an analogue or derivative thereof that binds TLR9. CpG oligodeoxynucleotides (or CpG ODN) are short single-stranded synthetic DNA molecules that contain a cytosine triphosphate deoxynucleotide followed by a guanine triphosphate deoxynucleotide. The “p” refers to the phosphodiester link between consecutive nucleotides, although some ODN have a modified phosphorothioate (PS) backbone instead. When these CpG motifs are unmethlyated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is constitutively expressed internally only in B cells and plasmacytoid dendritic cells (pDCs) in humans and other higher primates. However, extracellular expression of this receptor only happens in certain pathologies. Moreover, MDS progenitors, and in particular MDS stem cells (HSC), overexpress Toll-like receptor (TLR)-9 extracellularly, permitting development of a targeting approach using unmethylated CpG oligonucleotides linked to bioactive payloads for cellular delivery.

Synthetic CpG ODN differ from microbial DNA in that they have a partially or completely phosphorothioated (PS) backbone instead of the typical phosphodiester backbone and a poly G tail at the 3′ end, 5′ end, or both. PS modification protects the ODN from being degraded by nucleases such as DNase in the body and poly G tail enhances cellular uptake. The poly G tails form intermolecular tetrads that result in high molecular weight aggregates. Numerous sequences have been shown to stimulate TLR9 with variations in the number and location of CpG dimers, as well as the precise base sequences flanking the CpG dimers. This led to the creation of five unofficial classes or categories of CpG ODN based on their sequence, secondary structures, and effect on human peripheral blood mononuclear cells (PBMCs). The five classes are Class A (Type D), Class B (Type K), Class C, Class P, and Class S.

Bivalent Linker

The bivalent linker can be any molecule suitable to link a compound, polypeptide, or nucleic acid to a TTL (e.g., CpG ODN). Methods and compositions for conjugating biomolecules, such as polynucleotides, are disclosed in G. T. Hermanon, Bioconjugate Techniques (2^{nd} ed.), Academic Press (2008), which is incorporated by reference in its entirety for the teaching of these techniques.

In some embodiments, the bivalent linker is a non-nucleotidic linker. As used herein, the term “non-nucleotidic” refers to a linker that does not include nucleotides or nucleotide analogs. Typically, non-nucleotidic linkers comprise an atom such as oxygen or sulfur, a unit such as C(O), C(O)NH, SO, SO₂, SO₂NH, or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, SS, S(O), SO2, N(R¹)₂, NR¹, C(O), C(O)O, C(O)NH, —OPO₂O—, cleavable linking group, substituted or unsubstituted aryl, substituted or unsubstituted heteroaryl, substituted or unsubstituted heterocyclic; where R′ is hydrogen, acyl, aliphatic or substituted aliphatic.

In some embodiments, the bivalent linker comprises at least one cleavable linking group, i.e. the linker is a cleavable linker. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the linked components after transport to the desired target. The intended nature of the conjugation or coupling interaction, or the desired biological effect, will determine the choice of linker group. For example, the bivalent linker can comprise a photocleavable PC linker. In some embodiments, the oligonucleotide is cleavable by dicer to produce isolate individual siRNA from the oligonucleotide.

Additional examples of linkers include Hexanediol, Spacer 9, Spacer 18, 1′,2′-Dideoxyribose (dSc), and I-Linker.

Cytotoxic Agents

The cytotoxic agent of the disclosed CpG-lytic peptides can be any agent capable of killing a cancer cell, e.g., when internalized by the cell. For example, the cytotoxic agent may be a cytotoxic peptide (e.g., lytic peptide) or a radionuclide. Suitable cytotoxins are known to those skilled in the art and include plant and bacterial toxins, such as, abrin, alpha toxin, diphtheria toxin, exotoxin, gelonin, pokeweed antiviral protein, ricin, and saporin.

Many natural and synthetic peptides and proteins having cytolytic activity are known. Cytolytic peptides are also described as pore-forming peptides or cytolysins. Interactions of pore forming peptides with the surface of the membrane may be based on nonspecific electrostatic interactions of the positively charged peptide with negatively charged surface of cell membrane. These peptides are generally of cationic character, so that they are capable of electrostatic interactions with surfaces with predominantly negatively charged particles. Upon contact and interaction of a cytolytic peptide with lipids on the cell surface, and after penetration inside the cell with the lipids on the surface of the mitochondrial membrane, interruption of the continuity of the cell membrane occurs, followed by formation of small size transmembrane pores, by which leakage of the contents of the cytoplasm, including ions, outside the cell occurs, resulting in rapid and irreversible electrolyte imbalance in the cell, cell lysis and death. The interactions of pore-forming peptides with the surface of the membrane may also include interactions with specific receptors present on the surface.

Naturally occurring cytolytic peptides of bacterial, plant or mammalian origin capable of forming pores include cecropin A and B, aurein 1.2, citropin 1.1, defensin (HNP-2), lactoferricin B, tachyplesin, PR-39, cytolysins of Enterococcus faecalis, delta hemolysin, diphtheria toxin, cytolysin of Vibrio cholerae, toxin from Actinia equina, granulysin, lytic peptides from Streptococcus intermedius, lentiviral lytic peptides, leukotoxin of Actinobacillus actinomycetemcomitans, magainin, melittin, lymphotoxin, enkephalin, paradaxin, perforin (in particular the N-terminal fragment thereof), perfringolysin 0 (PFO/theta toxin) from Clostridium perfringens, and streptolysins.

There are also known synthetic cytolytic pore-forming peptides. They are often hybrids of natural cytolytic peptides fragments, such as a hybrid of a cecropin A fragment and a magainin 2 fragment or a hybrid of a cecropin A fragment and a melittin fragment. Other wellknown cytolytic synthetic peptides are described, for example, in Regen et al., Biochem. Biophys. Res. Commun. (199) 159:566-571, which is incorporated by reference for these peptides.

In some embodiment, the lytic peptide comprises an amino acid sequence selected from the group consisting of KIKMVISWKG (SEQ ID NO: 1; HYD1); AIAMVISWAG (SEQ ID NO:2; HYDE); AIKMVISWAG (SEQ ID NO:3; HYDE); AIKMVISWKG (SEQ ID NO:4; HYD2); AKMVISW (SEQ ID NO:5); AKMVlSWKG (SEQ ID NO:6); IAMVISW (SEQ ID NO:7); IAMVISWKG (SEQ ID NO:8); IKAVISW (SEQ ID NO:9); IKAVISWKG (SEQ ID NO: 10); IKMAISW (SEQ ID NO: 11); IKMAISWKG (SEQ ID NO: 12); IKMVASW (SEQ ID NO: 13); IKMVASWKG (SEQ ID NO: 14); IKMVIAW (SEQ ID NO: 15); IKMVIAWKG (SEQ ID NO: 16); IKMVISA (SEQ ID NO: 17); IKMVISAKG (SEQ ID NO: 18); IKMVISW (SEQ ID NO: 19); IKMVISWAG (SEQ ID NO:20); KMVISWKA (SEQ ID NO:21); IKMVISWKG (SEQ ID NO:22; HYD18; (-K)HYD1); ISWKG (SEQ ID NO:23); KAKMVISWKG (SEQ ID NO:24); KIAMVISWAG (SEQ ID NO:25; HYD7); KIAMVISWKG (SEQ ID NO:26); KIKAVISWKG (SEQ ID NO:27); KIKMAISWKG (SEQ ID NO:28); KIKMV (SEQ ID NO:29); KIKMVASWKG (SEQ ID NO:30); KIKMVI (SEQ ID NO:31; HYD16); KIKMVIA WKG (SEQ ID NO:32); KIKMVIS (SEQ ID NO:33; HYD15); KIKMVISAKG (SEQ ID NO:34); KIKMVISW (SEQ ID NO:35; HYD14); KIKMVISWAG (SEQ ID NO:36); KIKMVISWK (SEQ ID NO:37; HYD17; HYDl(-G)); KIKMVISWKA (SEQ ID NO:38); KMVISWKG (SEQ ID NO:39; HYD9); LSWKG (SEQ ID NO:40; HYD12); MVISWKG (SEQ ID NO:41; HYDlO); SWKG (SEQ ID NO:42; HYD13); VISWKG (SEQ ID NO:43; HYDI 1); WIKSMKIVKG (SEQ ID NO:44); KMVIXW (SEQ ID NO:45); IKMVISWXX (SEQ ID NO:46); and KMVISWXX (SEQ ID NO:47); wherein X is any amino acid (traditional or non-traditional amino acid). In another embodiment, the peptide consists of the identified amino acid sequence. In another embodiment, the peptide consists essentially of the identified amino acid sequence. The lytic peptide can comprise at least one D-amino acid. In some cases, each amino acid of the peptide is a D-amino acid.

In some embodiments, the cytotoxic agent is a pyrrolobenzodiazepine (PBD). Pyrrolobenzodiazepines (PBDs) are known in the art, some of which have the ability to recognise and bond to specific sequences of DNA. PBDs are of the general structure:

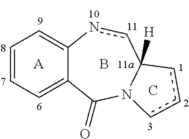

They differ in the number, type and position of substituents, in both their aromatic A rings and pyrrolo C rings, and in the degree of saturation of the C ring. In the B-ring there is either an imine (N═C), a carbinolamine(NH—CH(OH)), or a carbinolamine methyl ether (NH—CH(OMe)) at the N10-C11 position which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, InAntibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing.

In some embodiments, the cytotoxic agent is a functional nucleic acid, such as one that inhibits anti-apoptotic gene targets (e.g., Bcl-2, Bcl-xL, and Mcl-1), or promotes apoptotic gene targets (e.g., Bax, Bak, and Bcl-xS).

Functional nucleic acid molecules can be divided into the following categories, which are not meant to be limiting. For example, functional nucleic acids include antisense molecules, aptamers, ribozymes, triplex forming molecules, RNAi, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d}) less than or equal to 10⁻⁶, 10⁻⁸, 10⁻¹⁰, or 10⁻¹². A representative sample of methods and techniques which aid in the design and use of antisense molecules can be found in U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with K_{d}'s from the target molecule of less than 10⁻¹² M. It is preferred that the aptamers bind the target molecule with a K_{d} less than 10⁻⁶, 10⁻⁸, 10⁻¹⁰, or 10⁻¹². Aptamers can bind the target molecule with a very high degree of specificity. For example, aptamers have been isolated that have greater than a 10,000 fold difference in binding affinities between the target molecule and another molecule that differ at only a single position on the molecule (U.S. Pat. No. 5,543,293). It is preferred that the aptamer have a K_{d} with the target molecule at least 10, 100, 1000, 10,000, or 100,000 fold lower than the K_{d} with a background binding molecule. It is preferred when doing the comparison for a polypeptide for example, that the background molecule be a different polypeptide. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203; International Patent Application Nos. WO 9858058 by Ludwig and Sproat, WO 9858057 by Ludwig and Sproat, and WO 9718312 by Ludwig and Sproat) hairpin ribozymes (for example, U.S. Pat. Nos. 5,631,115, 5,646,031, 5,683,902, 5,712,384, 5,856,188, 5,866,701, 5,869,339, and 6,022,962), and tetrahymena ribozymes (for example, U.S. Pat. Nos. 5,595,873 and 5,652,107). There are also a number of ribozymes that are not found in natural systems, but which have been engineered to catalyze specific reactions de novo (for example, U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶, 10⁻⁸, 10⁻¹⁰, or 10⁻¹². Representative examples of how to make and use triplex forming molecules to bind a variety of different target molecules can be found in U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391:806-11; Napoli, C., et al. (1990) Plant Cell 2:279-89; Hannon, G. J. (2002) Nature, 418:244-51). Once dsRNA enters a cell, it is cleaved by an RNase III-like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200; Bernstein, E., et al. (2001) Nature, 409:363-6; Hammond, S. M., et al. (2000) Nature, 404:293-6). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309-21). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-74). However, the effect of iRNA or siRNA or their use is not limited to any type of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (UiTei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER® siRNA Construction Kit. Disclosed herein are any siRNA designed as described above based on the sequences for anti-apoptotic Bcl-2 member proteins, e.g., Bcl-2, Bcl-xL, and Mcl-1.

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits and Invitrogen's BLOCK-IT™ inducible RNAi plasmid and lentivirus vectors. Disclosed herein are any shRNA designed as described above based on the sequences for the herein disclosed inflammatory mediators.

Pharmaceutical Composition

Also disclosed is a pharmaceutical composition comprising a molecule disclosed herein in a pharmaceutically acceptable carrier. Pharmaceutical carriers are known to those skilled in the art. These most typically would be standard carriers for administration of drugs to humans, including solutions such as sterile water, saline, and buffered solutions at physiological pH. For example, suitable carriers and their formulations are described in Remington: The Science and Practice of Pharmacy (21 ed.) ed. PP. Gerbino, Lippincott Williams & Wilkins, Philadelphia, Pa. 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Methods of Treatment

Also disclosed is a method for treating a TLR9-expressing cancer, such as a myelodysplastic syndrome (MDS), in a subject by administering to the subject a therapeutically effective amount of the disclosed pharmaceutical composition. The method can further involve administering to the subject lenalidomide, or an analogue or derivative thereof. FIG. 4 identifies other cancers, such as lung and breast cancers, that have increased TLR9 expression. FIG. 5 shows TLR9 protein expression in a variety of tumor tissues. For example, cancers of the skin, esophagous, colon, rectum, liver, lung, and uterus have been shown to have increased TLR9 protein expression.

In some cases, the method further involves assaying a biopsy sample from the subject for TLR9 expression prior to treatment. This can be done using routine methods, such as immunodetection methods. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Parenteral administration of the composition, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

The compositions disclosed herein may be administered prophylactically to patients or subjects who are at risk for a TLR9-expressing cancer. Thus, the method can further comprise identifying a subject at risk for a TLR9-expressing cancer prior to administration of the herein disclosed compositions.

The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein. For example, effective dosages and schedules for administering the compositions may be determined empirically, and making such determinations is within the skill in the art. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. A typical daily dosage of the disclosed composition used alone might range from about 1 μg/kg to up to 100 mg/kg of body weight or more per day, depending on the factors mentioned above.

In some embodiments, the molecule is administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of molecule administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

### Definitions

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “CpG motif” refers to a nucleotide sequence, which contains unmethylated cytosine-guanine dinucleotide linked by a phosphate bond.

The term “CpG oligodeoxynucleotide” or “CpG ODN” refers to an oligodeoxynucleotide comprising at least one CpG motif and that binds TLR9.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies”, “intragenic”, etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “isolated polypeptide” refers to a polypeptide, which may be prepared from recombinant DNA or RNA, or be of synthetic origin, some combination thereof, or which may be a naturally-occurring polypeptide, which (1) is not associated with proteins with which it is normally associated in nature, (2) is isolated from the cell in which it normally occurs, (3) is essentially free of other proteins from the same cellular source, (4) is expressed by a cell from a different species, or (5) does not occur in nature.

The term “isolated nucleic acid” refers to a polynucleotide of genomic, cDNA, synthetic, or natural origin or some combination thereof, which (1) is not associated with the cell in which the “isolated nucleic acid” is found in nature, or (2) is operably linked to a polynucleotide to which it is not linked in nature.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “nucleic acid” refers to a polymeric form of nucleotides, either ribonucleotides or deoxynucleotides or a modified form of either type of nucleotide. The terms should also be understood to include, as equivalents, analogs of either RNA or DNA made from nucleotide analogs, and, as applicable to the embodiment being described, single-stranded (such as sense or antisense) and double-stranded polynucleotides.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “protein” (if single-chain), “polypeptide” and “peptide” are used interchangeably herein when referring to a gene product, e.g., as may be encoded by a coding sequence. When referring to “polypeptide” herein, a person of skill in the art will recognize that a protein can be used instead, unless the context clearly indicates otherwise. A “protein” may also refer to an association of one or more polypeptides. By “gene product” is meant a molecule that is produced as a result of transcription of a gene. Gene products include RNA molecules transcribed from a gene, as well as proteins translated from such transcripts.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “specifically deliver” as used herein refers to the preferential association of a molecule with a cell or tissue bearing a particular target molecule or marker and not to cells or tissues lacking that target molecule. It is, of course, recognized that a certain degree of nonspecific interaction may occur between a molecule and a non- target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A number of embodiments of the invention have been described. Nevertheless, it will be understood that various modifications may be made without departing from the spirit and scope of the invention. Accordingly, other embodiments are within the scope of the following claims.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Testing CpG-Lytic Peptides in MDS Mouse Models and Human Primary MDS BM Specimens

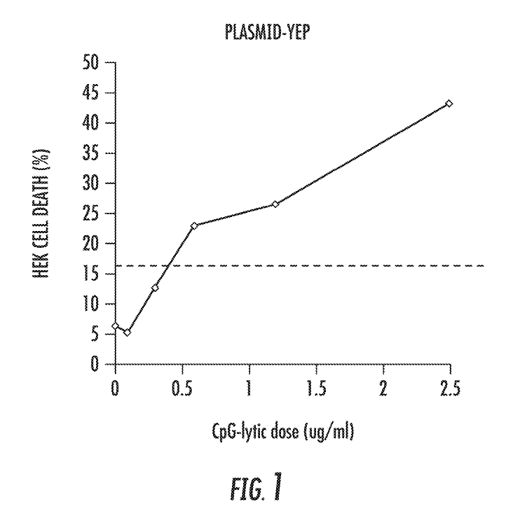

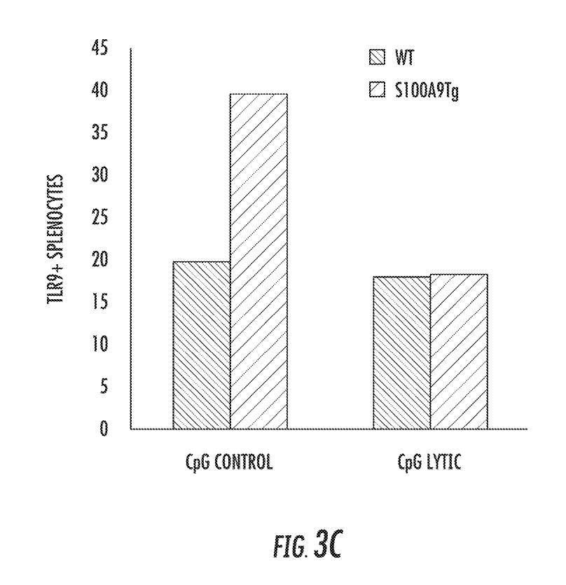

The underlying molecular mechanisms for MDS clonal expansion is diverse and the precise genes responsible for MDS clonal expansion have not been identified. This obstacle, however, can be overcome by using CpG-linked lytic peptides as shown in FIGS. 1 and 3A-3C. This approach is based on overexpression of plasma membrane TLR9 in the MDS clone versus healthy HSC and S100A9Tg mice. This can be combined with a lytic peptide, e.g., HYD1 (KIKMVISWKG, SEQ ID NO:1), for killing cancer cells. These lytic peptides are chemically linked with CpG and designed to efficiently deliver a payload through TLR9 receptor. They are dependent on internalization, and release of the lytic peptides from the CpG intracellularly to elicit their lytic activity. Therefore, small targeted CpG linked oncolytic peptides seek and destroy TLR9⁺ MDS cells (CD34⁺CD90⁺TLR9⁺) without harming normal HSCs (CD34⁺CD90⁺). The peptides are linear, alpha helical, cationic and they directly interact with negatively charged inner membranes resulting in disruption and cell death. Moreover, this has been shown to be effective in both BMMNCs from primary MDS patients and S100A9Tg mice (FIG. 3A-3C). These encouraging preclinical results validate the effect of these compounds in MDS in vitro and in in vivo animal models.

### Example 2: ¹⁸F Labeled CpG

A molecular PET imaging agent ¹⁸F labeled CpG to monitor in real time the in vivo targeting of TLR9 expressing MDS cells is designed. S100A9Tg mice provide a unique opportunity to test a reagent in vivo. The S100A9Tg mice are treated with ¹⁸F-CpG at Day 1 to obtain the background molecular PET image, before treatment with a test a reagent for two weeks (three times/week for two weeks). At this point the animals will be allowed to recover for 2 more days before infusion with ¹⁸F-CpG to monitor the decrease in malignant HSCs in the animal's BM by molecular-PET imaging analysis. The change in micro-PET/CT imaging observed in the BM and spleen reflects the specific targeted drug effect due to the decrease of TLR9⁺ MDS malignant clones. In comparison, the mice treated with control agent or ¹⁸F alone have no change in this population of cells and will serve as background controls. In addition, ¹⁸F-labeled scrambled CpG can be included as a control to corroborate the specificity of ¹⁸F-CpG as a vehicle of drug delivery. Alternatively, mice can be treated twice weekly with alternate schedules of drug and ¹⁸F-CpG in order to monitor the PK/PD of drug distribution, uptake and occupancy as well as the optimal dosage at the target site. In vitro labeling efficiency is characterized and the in vitro labeling efficiency of ¹⁸F labeled CpG optimized. Quality control and lipophilicity (log P) measurements are performed. In vitro studies include cell uptake and biostability measurements in plasma. The procedure for cGMP production and radiosynthesis is optimized. Tandem analysis includes several groups with varying doses of conjugate to assess the efficacy of the compound on MDS HSC reduction. In vivo and ex vivo studies include serum biostability, analysis of metabolism, biodistribution in normal tissues and bone marrow for the ¹⁸F-labeled probe; and ¹⁸F-labeled conjugates.

### Example 3: CpG Linked siRNA Conjugates Against BCL-2 Member of Proteins

The following CpG linked siRNA conjugates were made:

siRNA targeting BCL-2:
(SEQ ID NO: 49)
CpG-linker-5′-CCCUGUGGAUGACUGAGUA-3′;
siRNA targeting BCL-2XL:
(SEQ ID NO: 50)
CpG-linker-5′-GGAGUCAGUUUAGUGAUGU-3′;
siRNA targeting MCL-1:
(SEQ ID NO: 51)
CpG-linker-5′-GUAUCGAAUUUACAUUAGU-3′;
and
non-targeting control:
(SEQ ID NO: 52)
CpG-linker-5′-UGGUUUACAUGUCGACUAA-3′.

In each of the above, CpG had the sequence: 5′-TCCATGACGTTCCTGATGCT-3′ (SEQ ID NO:53).

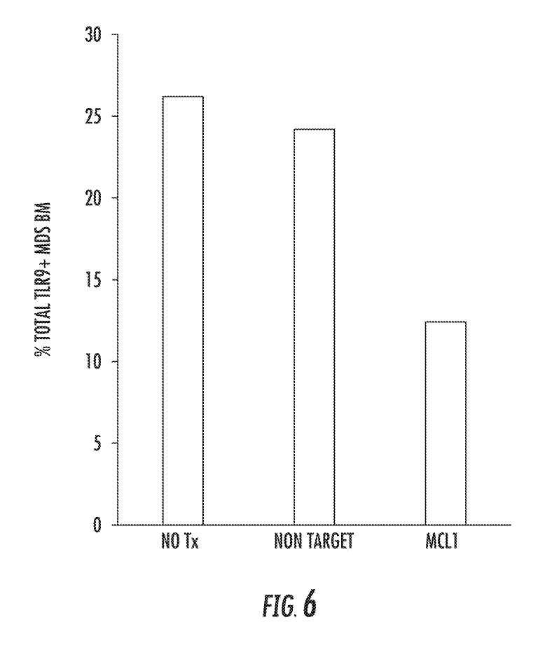

FIG. 6 shows an example of MDS BM patient specimen treated with the si-MCL-1 linked CpG demonstrating reduction of TLR9 positive cells after in vitro culture.

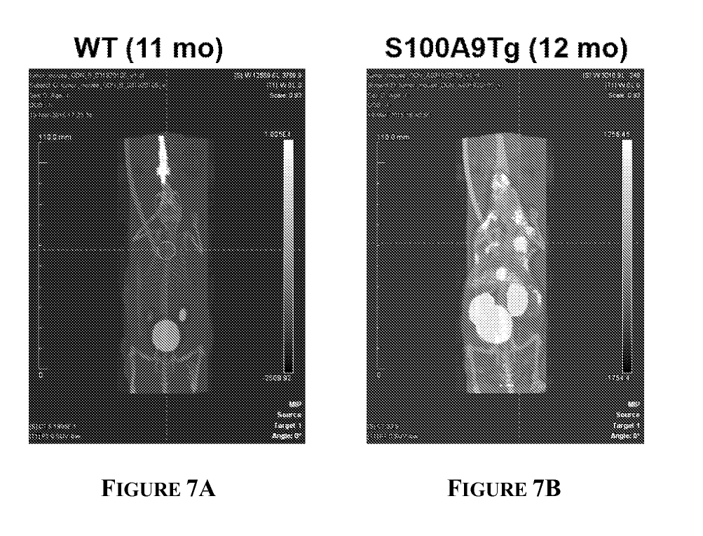

FIGS. 7A and 7B are CT and PET scans showing increased uptake of CpG-linked conjugates compared to normal controls, matching the in vitro data demonstrating that MDS malignant clones can be targeted by a CpG payload delivery approach.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A molecule comprising a toll like receptor-9 (TLR9) targeting ligand conjugated to a cytotoxic agent.

2. The molecule of claim 1, wherein the molecule is defined by the formula:
TTL-CA,
wherein “TTL” represents the TLR9 targeting ligand,
wherein “CA” represents cytotoxic agent, and
wherein “-” represents a bivalent linker.

3. The molecule of claim 1, wherein the cytotoxic agent comprises a lytic peptide.

4. The molecule of claim 1, wherein the TLR9 targeting ligand is an unmethylated CpG oligodeoxynucleotide, or an analogue or derivative thereof that binds TLR9.

5. The molecule of claim 3, wherein the lytic peptide comprises the amino acid sequence PNPNNNPNPN (SEQ ID NO:48), wherein “P” is any polar amino acid, and wherein “N” is any non-polar amino acid.

6. The molecule of claim 5, wherein the lytic peptide comprises the amino acid sequence KIKMVISWKG (SEQ ID NO:1).

7. The molecule of claim 1, wherein the cytotoxic agent comprises a functional nucleic acid that inhibits an anti-apoptotic gene target.

8. The molecule of claim 7, wherein the anti-apoptotic gene target comprises Bcl-2, Bcl-xL, Mcl-1, or a combination thereof.

9. A pharmaceutical composition comprising the molecule of claim 1 in a pharmaceutically acceptable carrier.

10. A method for treating a TLR9-positive cancer in a subject, comprising administering to the subject a therapeutically effective amount of the pharmaceutical composition of claim 9.

11. The method of claim 10, wherein the TLR9-positive cancer comprises a meylodysplastic syndrome (MDS).

12. The method of claim 11, wherein the TLR9-positive cancer comprises non-del(5q) MDS.

13. The method of claim 10, further comprising assaying a biopsy sample from the subject for TLR9 expression prior to treatment.


SEQUENCE LISTING (52 sequences)

SEQ ID NO: 1 - protein, 10 aa, Artificial Sequence
  Synthetic Construct
KIKMVISWKG

SEQ ID NO: 2 - protein, 10 aa, Artificial Sequence
  Synthetic Construct
AIAMVISWAG

SEQ ID NO: 3 - protein, 10 aa, Artificial Sequence
  Synthetic Construct
AIKMVISWAG

SEQ ID NO: 4 - protein, 10 aa, Artificial Sequence
  Synthetic Construct
AIKMVISWKG

SEQ ID NO: 5 - protein, 7 aa, Artificial Sequence
  Synthetic Construct
AKMVISW

SEQ ID NO: 6 - protein, 9 aa, Artificial Sequence
  Synthetic Construct
AKMVLSWKG

SEQ ID NO: 7 - protein, 7 aa, Artificial Sequence
  Synthetic Construct
IAMVISW

SEQ ID NO: 8 - protein, 9 aa, Artificial Sequence
  Synthetic Construct
IAMVISWKG

SEQ ID NO: 9 - protein, 7 aa, Artificial Sequence
  Synthetic Construct
IKAVISW

SEQ ID NO: 10 - protein, 9 aa, Artificial Sequence
  Synthetic Construct
IKAVISWKG

SEQ ID NO: 11 - protein, 7 aa, Artificial Sequence
  Synthetic Construct
IKMAISW

SEQ ID NO: 12 - protein, 9 aa, Artificial Sequence
  Synthetic Construct
IKMAISWKG

SEQ ID NO: 13 - protein, 7 aa, Artificial Sequence
  Synthetic Construct
IKMVASW

SEQ ID NO: 14 - protein, 9 aa, Artificial Sequence
  Synthetic Construct
IKMVASWKG

SEQ ID NO: 15 - protein, 7 aa, Artificial Sequence
  Synthetic Construct
IKMVIAW

SEQ ID NO: 16 - protein, 9 aa, Artificial Sequence
  Synthetic Construct
IKMVIAWKG

SEQ ID NO: 17 - protein, 7 aa, Artificial Sequence
  Synthetic Construct
IKMVISA

SEQ ID NO: 18 - protein, 9 aa, Artificial Sequence
  Synthetic Construct
IKMVISAKG

SEQ ID NO: 19 - protein, 7 aa, Artificial Sequence
  Synthetic Construct
IKMVISW

SEQ ID NO: 20 - protein, 9 aa, Artificial Sequence
  Synthetic Construct
IKMVISWAG

SEQ ID NO: 21 - protein, 8 aa, Artificial Sequence
  Synthetic Construct
KMVISWKA

SEQ ID NO: 22 - protein, 9 aa, Artificial Sequence
  Synthetic Construct
IKMVISWKG

SEQ ID NO: 23 - protein, 5 aa, Artificial Sequence
  Synthetic Construct
ISWKG

SEQ ID NO: 24 - protein, 10 aa, Artificial Sequence
  Synthetic Construct
KAKMVISWKG

SEQ ID NO: 25 - protein, 10 aa, Artificial Sequence
  Synthetic Construct
KIAMVISWAG

SEQ ID NO: 26 - protein, 10 aa, Artificial Sequence
  Synthetic Construct
KIAMVISWKG

SEQ ID NO: 27 - protein, 10 aa, Artificial Sequence
  Synthetic Construct
KIKAVISWKG

SEQ ID NO: 28 - protein, 10 aa, Artificial Sequence
  Synthetic Construct
KIKMAISWKG

SEQ ID NO: 29 - protein, 5 aa, Artificial Sequence
  Synthetic Construct
KIKMV

SEQ ID NO: 30 - protein, 10 aa, Artificial Sequence
  Synthetic Construct
KIKMVASWKG

SEQ ID NO: 31 - protein, 6 aa, Artificial Sequence
  Synthetic Construct
KIKMVI

SEQ ID NO: 32 - protein, 10 aa, Artificial Sequence
  Synthetic Construct
KIKMVIAWKG

SEQ ID NO: 33 - protein, 7 aa, Artificial Sequence
  Synthetic Construct
KIKMVIS

SEQ ID NO: 34 - protein, 10 aa, Artificial Sequence
  Synthetic Construct
KIKMVISAKG

SEQ ID NO: 35 - protein, 8 aa, Artificial Sequence
  Synthetic Construct
KIKMVISW

SEQ ID NO: 36 - protein, 10 aa, Artificial Sequence
  Synthetic Construct
KIKMVISWAG

SEQ ID NO: 37 - protein, 9 aa, Artificial Sequence
  Synthetic Construct
KIKMVISWK

SEQ ID NO: 38 - protein, 10 aa, Artificial Sequence
  Synthetic Construct
KIKMVISWKA

SEQ ID NO: 39 - protein, 8 aa, Artificial Sequence
  Synthetic Construct
KMVISWKG

SEQ ID NO: 40 - protein, 5 aa, Artificial Sequence
  Synthetic Construct
LSWKG

SEQ ID NO: 41 - protein, 7 aa, Artificial Sequence
  Synthetic Construct
MVISWKG

SEQ ID NO: 42 - protein, 4 aa, Artificial Sequence
  Synthetic Construct
SWKG

SEQ ID NO: 43 - protein, 6 aa, Artificial Sequence
  Synthetic Construct
VISWKG

SEQ ID NO: 44 - protein, 10 aa, Artificial Sequence
  Synthetic Construct
WIKSMKIVKG

SEQ ID NO: 45 - protein, 6 aa, Artificial Sequence
  Synthetic Construct
  misc_feature (5)..(5): Xaa can be any naturally occurring amino acid
KMVIXW

SEQ ID NO: 46 - protein, 9 aa, Artificial Sequence
  Synthetic Construct
  misc_feature (8)..(9): Xaa can be any naturally occurring amino acid
IKMVISWXX

SEQ ID NO: 47 - protein, 8 aa, Artificial Sequence
  Synthetic Construct
  misc_feature (7)..(8): Xaa can be any naturally occurring amino acid
KMVISWXX

SEQ ID NO: 48 - protein, 10 aa, Artificial Sequence
  Synthetic Construct
PNPNNNPNPN

SEQ ID NO: 49 - RNA, 19 nt, Artificial Sequence
  Synthetic Construct
cccuguggaugacugagua

SEQ ID NO: 50 - RNA, 19 nt, Artificial Sequence
  Synthetic Construct
ggagucaguuuagugaugu

SEQ ID NO: 51 - RNA, 19 nt, Artificial Sequence
  Synthetic Construct
guaucgaauuuacauuagu

SEQ ID NO: 52 - RNA, 19 nt, Artificial Sequence
  Synthetic Construct
ugguuuacaugucgacuaa